(19) United States
(12) Patent Application Publication
(10) Pub. No.: US 2024/0366065 A1
(43) Pub. Date: Nov. 7, 2024

(54) FLEXIBLE MINIATURE ENDOSCOPE

(71) Applicant: IPG PHOTONICS CORPORATION, Marlborough, MA (US)

(72) Inventors: Jeffrey Michael Treen, Nashua, NH (US); Olivier Traxer, Paris (FR); Isaac Ostrovsky, Wellesley, MA (US); Michael Barenboym, Boston, MA (US); Gregory Altshuler, Lincoln, MA (US); Ilya Yaroslavsky, North Andover, MA (US); Dmitri Boutoussov, Southborough, MA (US)

(73) Assignee: IPG PHOTONICS CORPORATION, Marlborough, MA (US)

(21) Appl. No.: 18/686,612

(22) PCT Filed: Sep. 9, 2022
(86) PCT No.: PCT/US2022/043047

§ 371 Date: Feb. 26, 2024

Related U.S. Application Data
(60) Provisional application No. 63/242,523, filed on Sep. 10, 2021.

(51) Int. Cl. A61B 1/00; A61B 1/055; A61B 1/07

(52) U.S. Cl. CPC A61B 1/00096; A61B 1/055; A61B 1/07

(57) ABSTRACT

An endoscopic device having reduced cross-section and a steering section of enhanced compliance. The device defines a smaller cross-section by eliminating need for pull wires and torsion sleeves. Use of a single fiber optic for steering opens up cross-sectional space to augment both irrigation and aspiration channels within a common catheter shaft. The single fiber optic may be utilized for “pulling” and/or “pushing” on the steering section, thereby providing unidirectional or bidirectional steering with a single fiber or fiber bundle. A distal steering section is configured to enhance compliance in response to the forces exerted by the single fiber optic. The enhanced compliance reduces the stoutness required of the fiber optic enabling a reduction in the size of the single fiber optic, thereby freeing up cross-section of the catheter for other uses. The enhanced compliance also enables tighter and more predictable articulation for better steering dexterity.

### RELATED APPLICATIONS

This application claims the benefit of U.S. Provisional Application No. 63/242,523, filed Sep. 10, 2021, the disclosure of which is hereby incorporated by reference herein in its entirety.

### FIELD OF THE DISCLOSURE

This application is directed generally to endoscopic devices and methods. More specifically, this application is directed to flexible, semi rigid, and rigid laser endoscopes for laser treatment of stones and tissues in humans and animals.

### BACKGROUND OF THE DISCLOSURE

Kidney stones affect 1 in 500 Americans each year, causing significant pain and healthcare expense. Surgical options for patients with symptomatic kidney stones include extracorporeal shock wave lithotripsy (ESWL), ureteroscopy, and percutaneous nephrolithotomy (PCNL). A person's renal anatomy, stone composition, and body habitus all play major roles in determining outcomes and therapeutic approach.

The role of ureteroscopy over the last ten years has increased due to reductions in the diameter of the flexible catheter shaft, enhanced steering and deflection capabilities, improvement of video-imaging, miniaturization of baskets and instruments, and advances in lithotripsy (stone breakage) with the advent of holmium (Ho) and thulium (Tm) lasers. Over 45% of all kidney stone surgeries in the United States are now done using small ureteroscope technology and lasers.

Ureteroscopy involves the use of a small flexible or rigid device called a ureteroscope to directly see and treat kidney stones. The ureteroscope device, which provides a video image and has small “working” channels, is inserted into the bladder and up the ureter until the kidney stone is encountered. The kidney stone can then either be broken up with laser energy that is transmitted via a fiber optic (laser fiber) to the target site, and/or extracted using small baskets. The advantage of this type of surgery is that body orifices are used for access, requiring no incisions.

Ureteroscopy is often a good option for small kidney stones in the ureter or kidney. Success rates for ureteroscopy for clearing smaller kidney stones is generally higher than that for shockwave lithotripsy. With laser ureteroscopy, kidney stones can be broken into small particles with maximum dimensions less than 1 millimeter or even less than 0.25 millimeter using laser settings optimized for the purpose. In this case, products of ablation can be removed with irrigation flows or after surgery due to natural outflow from kidney to bladder to provide stone free treatment results.

However, ureteroscopy does not always work well with very large kidney stones (e.g., with dimensions greater than 20 millimeters), as the large size necessitates long treatment times and can pose difficulties in removing the fragments of such stones. Furthermore, mid-sized stones or fragments (e.g., with maximum dimensions of 1 to 5 millimeters) can be difficult to treat with lasers using contact techniques. For example, ureteroscopes operating in contact mode can be subject to strong retropulsion effects, thereby requiring operation in a non-contact mode (e.g., “popcorning”), which is time consuming and does not guarantee stone free results. As a result, ureteroscopy does not always work well with very large kidney stones, as the large size necessitates long treatment time and can pose difficulties in removing the fragments of such stones. In such cases a percutaneous approach may be the best available option.

A device and attendant techniques that mitigate or resolve these disadvantages of ureteroscopy would be welcomed.

### SUMMARY OF THE DISCLOSURE

Various embodiments of the disclosure present endoscopic surgical instruments and methods that mitigate certain shortcomings of conventional ureteroscopy by providing a miniature endoscope having a reduced cross-section relative to conventional ureteroscopes, as well as enhanced steering capabilities that make the disclosed device more agile. The reduced cross-section creates less discomfort during treatments that require extended time periods, for example when removing larger kidney stones. The increased agility makes it easier to track or “chase” stone during the treatment, thereby decreasing the treatment time and providing a higher probability of stone free results.

The present disclosure builds on International Patent Application Publication No. WO 2020/150713 to Altshuler et al. (“Altshuler”), the disclosure of which is hereby incorporated by reference herein in its entirety except for express definitions and patent claims contained therein. Altshuler addresses several of the shortcomings of laser ablation ureteroscopy for removal of large kidney stones. The present disclosure represents improvements to certain embodiments of Altshuler.

Various embodiments of the disclosure present a catheter cross-section having a more compact radial profile than conventional endoscopes by eliminating need for pull wires and torsion sleeves. The use of fiber optics, and in particular a single fiber optic, to perform the steering function opens up cross-sectional space in the scope and specifically in a head portion of the catheter to allow use of both irrigation and aspiration channels within a common catheter shaft. In some embodiments, utilization of a single fiber optic wherein both “pulling” and “pushing” of the catheter head is facilitated for enhanced bidirectional steering with a single illumination fiber. This enables all the functions of the catheter—illumination, imaging, irrigation, aspiration, and ablation—within a cross-sectional dimension that is in a range of about two millimeters. Cross-sectional dimensions in this range can enable ureteroscopic removal of body stones without subjecting the patient to a general anesthesia.

To facilitate the use of a single fiber optic for steering, various embodiments of the disclosure include a distal end steering section that reduces the resistance (i.e., enhances the compliance) of the steering section in response to the forces exerted by the pushing/pulling of the single fiber optic. The enhanced compliance reduces the stoutness required of the fiber optic, particularly when in compression during pushing, where buckling of the single fiber optic is a consideration. The reduced stoutness requirement enables the steering operation to be completed with a single fiber optic of smaller cross-section than would be required for a less-compliant steering section. The enhanced compliance also concentrates the bending of the catheter at the steering section for tighter and more predictable articulation, thereby enhancing the dexterity of the steering operation with less required force.

In some embodiments, the resilience of various components passing through the cross-section exert a sufficient lateral bias on the distal end steering section to passively return the distal end steering section to a neutral orientation. Such components may include, alone or in combination, a separate fiber optic (e.g., a laser fiber optic), a sleeve surrounding the steering section, a spine of the distal end steering section, and the steering fiber optic itself. In some embodiments, an auxiliary biasing element may be implemented, for example imbedded in or otherwise integral with the spine of the distal end steering section, to enhance the lateral biasing. Passive return of the dislat steering section to the neutral orientation enables unidirectional steering without need for actively pushing the distal end steering section into the neutral orientation.

Structurally, an endoscopic device is disclosed, comprising a steering section including a plurality of segments arranged sequentially along a central axis, the plurality of segments being separated at a first lateral side of the steering section to define a plurality of gaps therebetween. A fiber optic extends to a distal end portion of the steering section. Placing the fiber optic in tension causes the steering section to deflect in a first lateral direction. The plurality of segments may be joined at a second lateral side of the steering section. In some embodiments, the fiber optic is anchored proximate the distal end portion of the steering section, and may be an illumination fiber optic.

In some embodiments, the endoscopic device includes a distal head portion attached to the distal end portion. The distal head portion may include a base and a transparent cap. In some embodiments, the fiber optic is anchored to the base of the distal head portion. The steering section may define a guide passage proximate the first lateral side, the fiber optic being disposed in the guide passage. In some embodiments, each of the plurality of segments defines a guide passage segment to define the guide passage, the guide passage segments being concentric about a guide axis, the fiber optic passing through the guide passage segments along the guide axis. The fiber optic may be a single fiber optic that passes through the guide passage segments. In some embodiments, the single fiber optic defines an oblong cross-section.

In some embodiments, the steering portion defines a first working channel and a second working channel, the first working channel being breached to define the plurality of gaps. The second working channel may be adjacent the second lateral side. In some embodiments, the second working channel being continuous through the steering section. The plurality of segments may be surrounded by a flexible sleeve, which may be anchored to the base of the head portion, and/or anchored to a proximal portion of the steering section.

For various embodiments of the disclosure, placing the fiber optic in compression causes the steering section to deflect in a second lateral direction. The first lateral direction may be opposite the second lateral direction. In some embodiments, the first lateral side is in the first lateral direction from the central axis, and the second lateral side is in the second lateral direction from the central axis.

### BRIEF DESCRIPTION OF THE DRAWINGS

FIG. 1 is a schematic of an endoscopic system for laser lithotripsy according to an embodiment of the disclosure;

FIG. 2 is a partially exploded perspective view of a distal portion of the catheter having a steering section according to an embodiment of the disclosure;

FIG. 3 is an end view of the distal portion of the catheter of FIG. 2 as assembled according to an embodiment of the disclosure;

FIG. 3A is an end view of an alternative distal portion for the catheter of FIG. 2 as assembled according to an embodiment of the disclosure;

FIG. 4 is a partial, sectional view of the distal portion of the catheter at plane IV-IV of FIG. 3 according to an embodiment of the disclosure:

FIG. 4A is a partial, sectional view of the distal portion of the catheter at plane IVA-IVA of FIG. 3A according to an embodiment of the disclosure;

FIG. 5 is an elevational view of the distal portion of the catheter of FIG. 2 in a neutral orientation according to an embodiment of the disclosure;

FIG. 6 is an elevational view of the distal portion of FIG. 5 in a fully collapsed configuration according to an embodiment of the disclosure; and

FIG. 7 is an elevational view of the distal portion of FIG. 5 in a fully expanded configuration according to an embodiment of the disclosure.

### DETAILED DESCRIPTION OF THE DRAWINGS

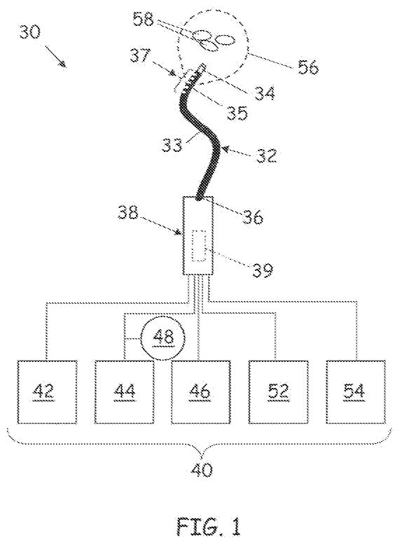

Referring to FIG. 1, an endoscopic system for laser lithotripsy is schematically depicted according to an embodiment of the disclosure. The endoscopic system 30 includes a catheter 32 having a near end 36 coupled to a handle 38 and a distal portion 35 that includes a distal head portion 34 and a steering section 37. The catheter 32 includes a catheter shaft 33 that may be flexible (depicted). The handle 38 may house a steering mechanism 39 that is coupled to the distal head portion 34. The handle 38 integrates various external components or systems 40 for control and delivery to the distal head portion 34 via the catheter 32. The external systems 40 may include an irrigation system 42, a suction or aspiration system 44, an ablation laser system 46, an illumination system 52, and a visualization system 54. Some of the components of the endoscopic system 30 may be partially or completely integrated into the handle 38, the catheter 32, or the distal head portion 34. The handle 38, for example, may include control mechanism of the aspiration and irrigation systems 42 and 44, and a mechanism for adjusting position of the distal end of the laser fiber, as well as other components. The mechanism of fiber positioning may include a clamp (not depicted) that can be engaged once a distal extremity of the laser fiber is in the desired position. Clamping the laser fiber fixes the position of the distal extremity, typically with an accuracy in the range of 0.05 to 0.1 millimeters. The direction from the catheter shaft 33 to the distal head portion 34 along a central axis 110 is herein referred to as the distal direction 50. The direction opposite the distal direction 50 is herein referred to as the proximal direction 51.

Functionally, the steering mechanism 39 enables articulation of the steering section 37 at the distal portion 35 of the catheter 32, for routing through body vessels of the patient to a target zone 56 and for alignment of the distal head portion 34 to hone in on individual body stones 58 within the target zone 56. The steering section 37 enables the distal portion 35 of the catheter 32 to articulate without undue stress and strain and distortion. The illumination system 52 generates visible light that is delivered to the target zone 56 for illumination of the body stones 58 and surrounding tissue, for example stones within a kidney, ureter or bladder. The ablation laser system 46 includes, for example, a Thulium or Holmium fiber or solid state laser, for delivering laser energy to the target zone 56 for ablation and break up of body stones 58. Delivery of the laser energy may be accomplished using a laser fiber, for example, silica or other optical fiber material. The irrigation system 42 provides pressurized irrigation fluid for cooling of the target zone 56 and for moving fragments of body stones 58 within the target zone 56. The aspiration system 44 draws liquid medium away from the target zone 56, including particles from the body stones 58 that may be suspended in the medium. In some embodiments, the aspiration system 44 includes a pressure sensor 48 that monitors the aspiration pressure. Pressure sensors may also be utilized to monitor the irrigation pressure.

Herein, “body stones” encompass any stone that is produced by the human body, including kidney stones and ureteral stones, as well as species thereof including calcium stones, uric acid stones, struvite stones, and cysteine stones. “Body stones” may also include stones found in or formed by other organs of the body, for example, bladder stones, gallbladder stones, prostate stones, pancreas stones, saliva gland stones, and belly stones. The present disclosure describes, but in general is not limited to, systems and techniques for breakup of kidney and ureteral stones. In view of this disclosure, those of skill in body stone therapies will recognize the application of various aspects disclosed herein for the remediation of body stones other than kidney and ureteral stones as well as for treatment of hard and soft tissues.

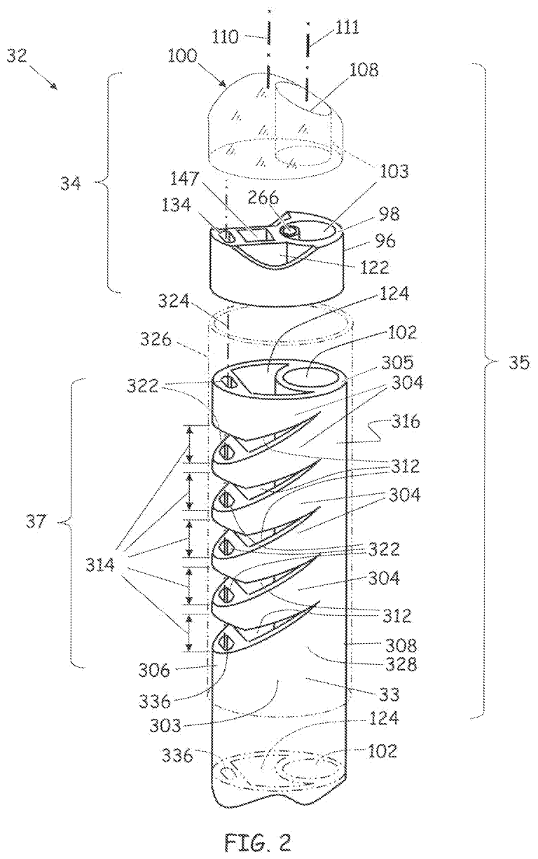

Referring to FIGS. 2, 3, and 4, the steering section 37 at the distal portion 35 of the catheter 32 is depicted according to an embodiment of the disclosure. In some embodiments, the steering section 37 includes a plurality of segments 304 that define and extend from a proximal portion 303 to a distal end portion 305 of the steering section 37. Herein, the proximal portion 303 of the steering section 37 is a region of the catheter 32 that is adjacent and proximal to a proximal-most of the plurality of gaps 312, described below.

The plurality of segments 304 are separated at a first lateral side 306 of the catheter shaft 33 and may be joined to each other at a second lateral side 308. The separation of the plurality of segments 304 defines a plurality of gaps 312 between the plurality of segments 304, each of the plurality of gaps 312 defining a maximum gap dimension 314 on the first lateral side 306. The second lateral side 308 where the plurality of segments 304 are joined may be characterized as a spine 316 of the steering section 37. In some embodiments, the spine 316 is diametrically opposed to the maximum gap dimensions 314 of the gaps 312 (depicted). In some embodiments, the steering section 37 defines working channels 102 and 124. The working channel 124 may breached to define the plurality of gaps 312 to provide the segmentation. The plurality of segments 304 may be formed around but not breach the working channel 102, to preserve the integrity of the working channel 102 as a continuous passage through the steering section 37.

In some embodiments, the plurality of segments 304 each define a plurality of guide passage segments 322 (one guide passage segment for each of the plurality of segments 3041 that is proximate the first lateral side 306 of the catheter shat 33. The plurality of guide passages 322 may define and be concentric about a guide axis 324. An illumination fiber optic 132 defining a cross-section 133 passes through the plurality of guide passages 322 and into an illumination fiber optic port 134. The illumination fiber optic port 134 may be defined at a base 96 of the distal head portion 34 (depicted). Alternatively, the illumination fiber optic port may be defined at the distal end portion 305 of the steering section 37.

In the depicted embodiment, the guide passages 322 are of oblong shape to accommodate a fiber optic 132 or fiber optic bundle of oblong cross-section. Guide passages and fiber optic cross-sections of other geometries (e.g., circular) may also be utilized. The illumination fiber optic 132 and each of the plurality of guide passages 322 may be dimensioned for a close sliding fit relative to each other. Herein, a “close sliding fit” is understood as a fit that enables sliding between components without noticeable play.

In some embodiments, the steering section 37 is surrounded by a sleeve 326 (depicted in phantom in FIG. 2) that extends over an exterior surface 328 of the steering section 37. The sleeve 326 may be anchored to the distal base 96 of the distal head portion 34, and may also be anchored to the proximal portion 303 of the steering section 37. The anchoring of the sleeve 326 may provide seal regions 334 that prevent liquids from seeping between the sleeve 326 and the exterior surface 328 of the steering section. In some embodiments, the catheter shaft 33 defines a guide lumen 336 that extends from the proximal portion 303 of the steering section 37 through the near end 36 of the catheter 32. The guide lumen 336 may be substantially aligned with the guide axis 324.

The sleeve 326 may be fabricated from a high elasticity membrane material, enabling the sleeve 326 to conform to the arcuate shape of the steering section 37 when flexed. Examples include thermoplastic elastomers such as PEBAX®, which has an elastic modulus of approximately 0.145 gigapascal. In some embodiments, the thickness of the sleeve 326 is in a range from 50 to 100 μm inclusive. In assembly, the sleeve 376 is cut to length, slid over the steering section 37 and heated to temperature (e.g., 80° C. to 120° C.).

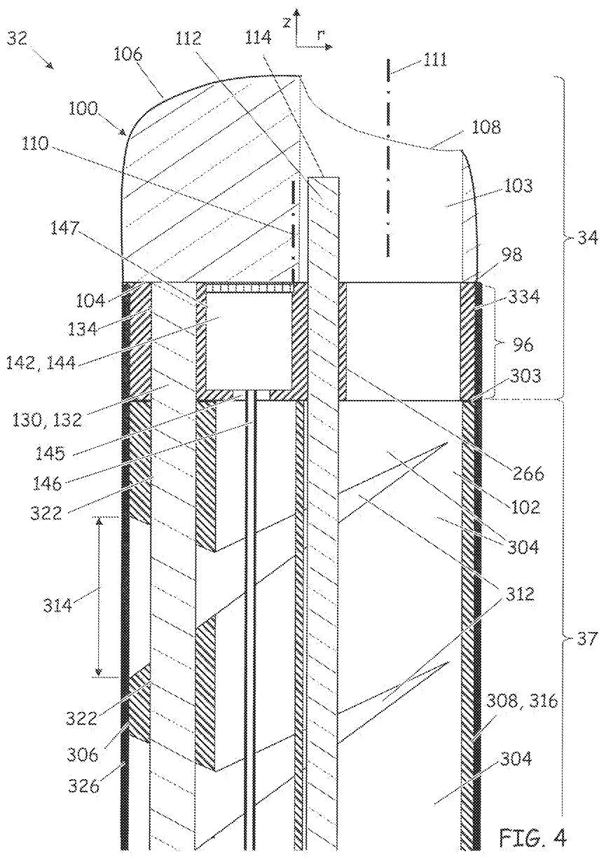

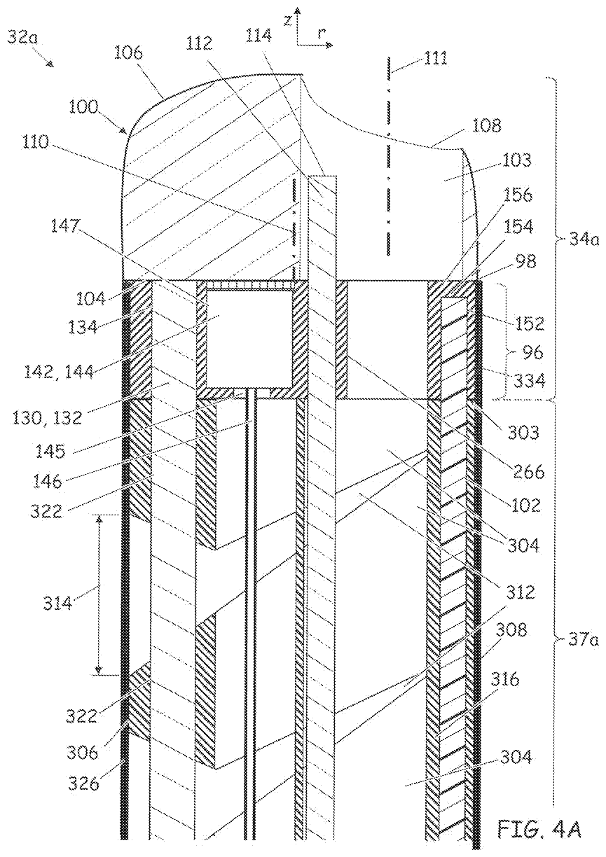

The distal head portion 34 may include the base 96. The base 96 may also be characterized as a distal tip or termination of the steering section 97. In some embodiments, the base 96 is formed separate from the catheter shaft 33 and affixed thereto, as depicted in FIGS. 2 and 4. In other embodiments, the base 96 is unitary with the catheter shaft 33 (not depicted). In some embodiments, a transparent cap portion 100 is secured to a distal face 98 of the base 96. The transparent cap portion 100 includes a proximal face 104 and a distal face 106. The transparent cap portion 100 is fabricated from a material appropriate for transmitting visible light and may include a low absorptivity and high damage threshold at the operating wavelengths of the ablation laser system 46. Non-limiting example materials for the transparent cap 100 include sapphire, quartz, optical ceramic, and mineral or organic glass. In some embodiments, the refractive index of the transparent cap 100 is about 1.31 to 1.35, to approximately match the refractive index of the liquid medium (substantially water). In some embodiments, the base 96 may be fabricated from the same transparent material as the transparent cap 100.

In some embodiments, the distal head portion 34 contains an illuminator 130. The illuminator 130 may be the distal end of an illumination fir lighting fiber optic 132 (depicted) or a fiber optic bundle (not depicted) for transmitting light in the visible spectrum. The illuminator 130 is operatively coupled to the illumination system 52 at the handle 38. Herein, the illuminator 130 is represented by the single fiber optic 132, but it is understood that a single fiber optic bundle may be used in place of the single fiber optic 132. The illumination fiber optic 132 passes through an illumination fiber optic port 134 formed in the base 96 of the distal head portion 34 and may extend into the transparent cap 100. The illumination fiber optic 132 acts as an optical waveguide.

In some embodiments, the illumination fiber optic 132 is mechanically affixed to the distal head portion 34 (e.g., with an adhesive), for example, to the illumination fiber optic port 134 or the transparent cap 100 or both. The distal head portion 34 is thereby coupled to the steering mechanism 39 of the handle 38 via the illumination fiber optic(s) 132. The coupling and routing of the illumination fiber optics 132 so arranged enables the illumination fiber optic(s) 132 to also serve as a pulling linkage or a push-pull linkage for steering of the distal head portion 34, thereby negating the need for separate pull wires and the connectors associated with coupling them to the distal head portion 34.

The distal head portion 34 defines a working channel 102 that passes through the base 96 of the distal head portion 34 and through the proximal face 104 and the distal face 106 of the transparent cap portion 100. The working channel 102 defines a mouth 108 at the distal face 106, the mouth 108 being concentric about a working port axis II. Herein, a “working channel” may serve as an irrigation channel, an aspiration channel, or both. Working channels as used herein may optionally be configured to accommodate working objects such as laser fibers and baskets. The inner diameter of the working port 103 may be in the range from 0.5 to 1.5 millimeters inclusive for flexible catheters utilizing a 0.05 millimeter core laser fiber. In one example, the working channel 102 serves as an aspiration port, in which case the mouth 108 and working channel define an aspiration inlet. The working channel 102 extends through the catheter 32 and may be coupled, tor example, to the aspiration system 44 at the handle 3. The working channel 102 includes a working port 103 that is formed in and passes through the distal head portion 34 and defines the mouth 108.

In some embodiments, the distal head portion 34 defines an oblong cross-section 167 (depicted) having a major axis 171 and a minor axis 169 and corresponding outer dimensions OD1 and OD2. Herein, an “oblong” cross section has a major dimension (011) and a minor dimension (OD2) that are perpendicular to each other and intersect at the central axis 110. The major dimension OD1 is the greatest dimension of the oblong cross section 167 that passes through the central axis 110. The minor dimension OD2 is a dimension that is perpendicular to the major dimension OD1 at the central axis 110 and is less than the major dimension OD1. The minor dimension OD2 may be, but isn't necessarily, the minimum dimension of the cross-section 167. In some embodiments, the outer dimension OD1 of the oblong cross-section 167 is in a range of 1.7 to 3.2 millimeter inclusive; in some embodiments, the outer dimension OD1 is in a range of 1.7 millimeters to 2.6 inclusive; in some embodiments, the outer dimension OD1 is in a range of 2.2 to 2.5 millimeters inclusive. In some embodiments, the outer dimension OD2 of the cross-section 167 is in a range of 1.7 to 2.5 millimeters inclusive; in some embodiments, the outer dimension OD2 is in a range of 1.7 to 2.0 millimeters.

In some embodiments, the distal head portion 34 includes a round cross-section (not depicted) that defines and is concentric about a central axis 110, the round cross-section having a diameter of about 2 millimeters. In some embodiments, the maximum diameter is in a range of 1.5 to 3 millimeters inclusive; in some embodiments, the maximum diameter is in a range of 1.8 to 2.5 millimeters inclusive; in some embodiments, the maximum diameter is in a range of 2 to 2.5 millimeters inclusive.

A laser fiber optic 112 for transmitting ablative laser energy is disposed in the working channel 102, a distal extremity 114 of the laser fiber optic 112 being positioned proximate the distal face 106 of the transparent cap portion 100, and a proximal end of the laser fiber optic 112 being coupled to the ablation laser system 46 via the handle 38. A core diameter of the laser fiber optic 112 may be in a range of 0.05 to 0.4 millimeters for a catheter having a flexible shaft, in some embodiments, the position of the distal extremity 114 of the laser fiber optic 112 can be controlled, for example, within a range of +/−5 millimeter inclusive relative to the distal face 106 of the transparent cap portion 100, where “+” and “−” refer respectively to the distal and proximal directions along the working port axis 111.

In some embodiments, the distal head portion 34 includes an imaging receiver 142, which may include image-forming optics defining a field of view for the endoscopic system 30. The imaging receiver 142 may be an imaging device 144 (depicted), such as a complementary metal oxide semiconductor (CMOS) sensor (including a semiconductor chip, imaging optics, and supporting electronics) or a charge-coupled device (CCD) camera sensor. In some embodiments, the imaging face the imaging receiver 142 is from 0.5×0.5 millimeter to 1.5×1.5 millimeter. An example of the described CMOS image sensor is the NANEYE 2D supplied by AWABA CMOS Image Sensors of Argau, Switzerland. See https://ams.com/naneye, last visited Jan. 16, 2020.

The imaging device 144 may include a cable 146 that extends through the catheter 32 and may be coupled to the visualization system 54 at the handle 38. The cable 146 may be routed through a cable port 145 defined by the base 96 of the distal head portion 34. In some embodiments, the imaging device 144 is disposed in a recess 147 at the distal face 98 of the base 96. Imaging devices 144 may define a viewing angle that is ±45 degrees of normal. Optionally, the imaging receiver 142 is a distal end of an optical system and imaging fiber optic (not depicted) which extends through the catheter 32 and is coupled to the visualization system 54 at the handle 38. The distal face 106 of the transparent cap 100 may be flat, rounded (depicted) or, alternatively, shaped as a lens for imaging onto the imaging receiver 142.

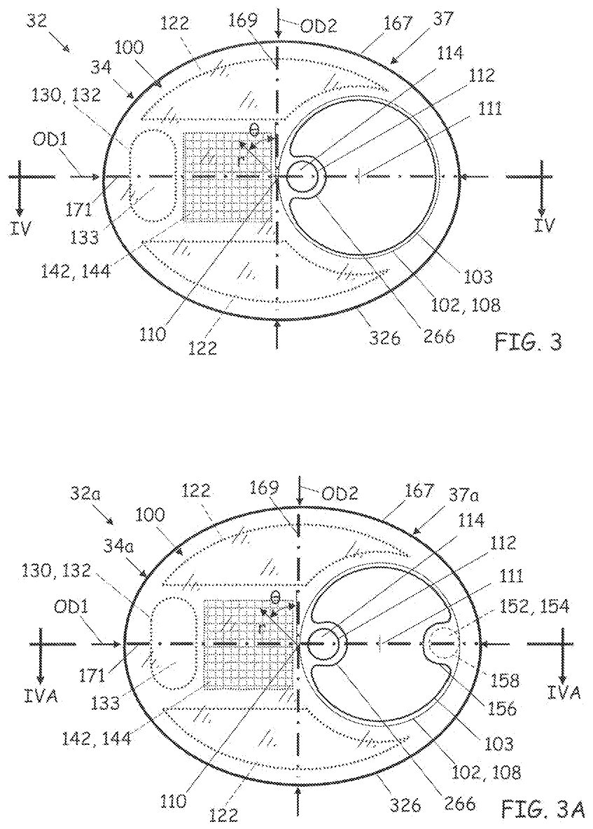

Referring to FIGS. 3A and 4A, a catheter 32a having an alternative distal head portion 34a and an alternative steering section 37a is depicted according to an embodiment of the disclosure. The catheter 32a may include some or all of the same components and attributes as the catheter 32, which are depicted in FIGS. 3A and 4A with same-labeled reference characters. The steering section 37a includes a resilient spine member 152 that extends along the working port 103 substantially parallel to the working port axis 111. In some embodiments, the resilient spine member 152 is imbedded in the spine 316 of the steering section 37a. Alternatively or in addition, a distal end 154 of the resilient spine member 152 may be terminated at a mount 156 included on the base 96 of the catheter 32. In some embodiments, the mount 156 defines a lumen or socket 156 for receiving the resilient spine member 152. The resilient spine member 152 may be anchored to the base 96 of the distal head portion 34, for example with an adhesive. In some embodiments, the resilient spine member 152 extends from the base 96 of the distal head portion 34 or otherwise proximate the distal end portion 305 to the proximal portion 303 of the steering section 37a.

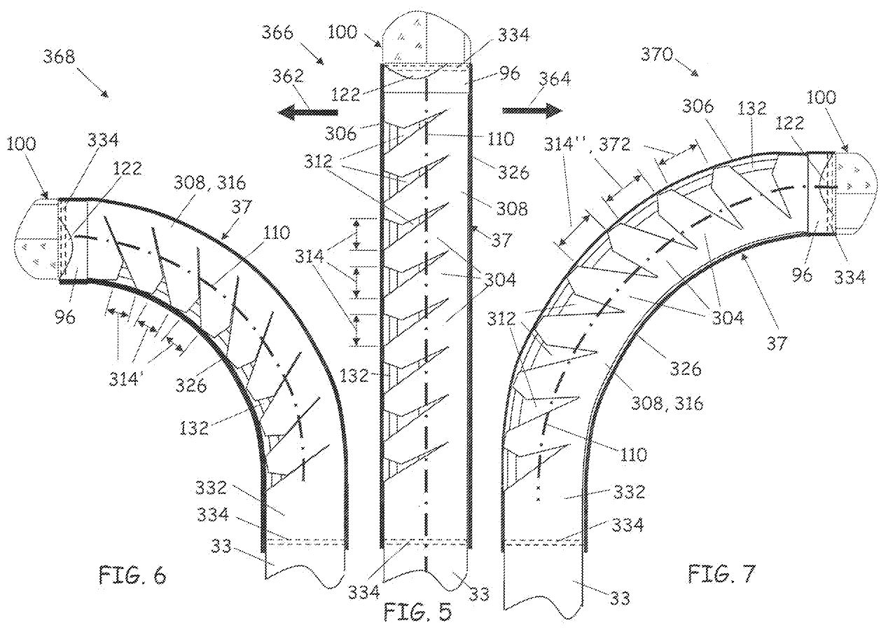

Referring to FIGS. 5 through 7, operation the steering section 37 is depicted according to an embodiment of the disclosure. In some embodiments, the steering section 37 can be deflected bilaterally. Herein, “bilateral” and its derivative terms refer to deflections in two different lateral directions 362 and 364 relative to the central axis 110, as depicted in FIGS. 5 through 7. These figures depict the steering section 37 in a neutral orientation 366 (FIG. 5), a fully collapsed orientation 368 (FIG. 6), and a fully expanded orientation 370 (FIG. 7). The “neutral” orientation refers to the state of the catheter 32 when there is no force exerted on the steering section 37 by the handle 38 via the steering mechanism 39. The “fully collapsed” orientation 368 is effected when the plurality of gaps 312 between the plurality of segments 304 are drawn together to the maximum extent, for example by a pulling stroke limit of the steering mechanism 39 or by drawing the plurality of segments 304 into seating contact with each other. The “fully expanded” orientation 370 is effected when the plurality of gaps 312 between the plurality of segments 304 are separated to the maximum extent, for example by a pushing stroke limit of the steering mechanism 39. The lateral directions 362 and 364 may be opposing (depicted). In some embodiments, the steering section 37 deflects the central axis 110 as much as 90 degrees in either lateral direction 362, 364 from the neutral orientation 366, for a total angular deflection range of up to 180 degrees from the fully collapsed orientation 368 to the fully expanded orientation 370. In some embodiments, the total angular deflection range is up to 270 degrees.

Functionally, the steering section 37 of the catheter shaft 33 may enable bilateral deflection with respect to the neutral orientation 366 using a single illumination fiber optic 132. Optionally, instead of a single fiber optic 132, a single fiber optic bundle (not depicted) may be implemented. When the single illumination fiber optic 132 is put in tension (i.e., is “pulled” through the catheter shaft 33), the distal head portion 34, to which the single illumination fiber optic 132 is anchored, is pulled proximally toward the proximal portion 303 of the steering section 37. The plurality of segments 304 of the steering section 37 along the first lateral side 306 are drawn together to define maximum gap dimensions 314′ of the plurality of gaps 312 (FIG. 6) that are reduced relative to the maximum gap dimensions 314 of the neutral orientation 366. Meanwhile, the portions of the plurality of segments 304 that define the spine 316 of the steering section 37 maintain substantially the same dimensions. The effect is to cause the steering section 37 to arc in the first lateral direction 362 relative to the neutral orientation 366. When the single illumination fiber optic 132 is put in compression (i.e., is “pushed” through the catheter shaft 33), the distal head portion 34 is pushed distally away from the proximal portion 303 of the steering section 37, such that the plurality of segments 304 of the steering section 37 along the first lateral side 306 are further separated to define maximum gap dimensions 314″ of the plurality of gaps 312 (FIG. 7) that are increased relative to the maximum gap dimensions 314 of the neutral orientation 366. Meanwhile, the portions of the plurality of segments 304 that define the spine 316 of the steering section 37 again maintain substantially the same dimensions. The effect is to cause the steering section 37 to are in the second lateral direction 364 relative to the neutral orientation 366. During the pulling and pushing operations, the close sliding fit between the single illumination fiber optic 132 and the plurality of guide passages 322 enable the plurality of segments 304 to be repositioned along the single illumination fiber optic 132 as the segments 304 are reoriented along the central axis 110.

Routing the single illumination fiber optic 132 through the plurality of segments 304 also prevents column buckling of the single illumination fiber optic 132 due to the compression forces encountered during the pushing operation of FIG. 7. During the pushing operation, the compression forces exerted on the single illumination fiber optic 132 are caused by, for example, the stretching of the sleeve 326, the bending of the spine 316, and sliding or frictional resistance against the body cavities of the patient. The so-called “critical force” required to cause column buckling is inversely proportional to a length of the column, as well as being proportional to the cross-sectional moment of inertia and the elastic modulus of the column. See. e.g., Budynas, “Advanced Strength and Applied Stress Analysis,” pp. 92-96, McGraw-Hill © 1977, the disclosure of which is hereby incorporated by reference herein except for express definitions contained therein. For the steering section 37 and illumination fiber optic 132, the column length may be approximated by a maximum unsupported length 372 of the single illumination fiber optic 132 between adjacent segments of the plurality of segments 304 (FIG. 7). Because the critical force required for column buckling is at a minimum when the unsupported length is at a maximum, the configuration of interest for preventing column buckling is the fully expanded orientation 370, where the unsupported length 372 is the longest. The maximum unsupported length 372 may correspond approximately to the maximum gap dimensions 314″ in the fully expanded orientation 370. Accordingly, the steering section 37 may be designed to prevent column buckling of the single illumination fiber optic 132 in the fully expanded configuration 370.

In some embodiments, the resilience of various components of the steering section 37 provide a resilience that is sufficient to laterally bias the distal end steering section 37 for passive return of the steering section 37 to the neutral orientation 366. Such components may include, alone or in combination, the laser fiber optic 112, the sleeve 326 surrounding the distal end steering section 37, the spine 316 of the distal end steering section 37, and/or the illumination fiber optic 132 itself. For embodiments that implement the auxiliary biasing member 152, passive return of the distal end steering section 37a to the neutral orientation 366 is enhanced by the additional lateral biasing. In such embodiments, return to the neutral orientation 366 may require little or no pushing with the fiber optic 132 to effect the neutral orientation 366.

The passive or nearly passive return of the distal end steering section 37, 37a enables unidirectional steering of the catheter 32, 32a. Unidirectional steering is characterized by the neutral orientation 366 (FIG. 5) and the fully collapsed orientation 368 (FIG. 6) as well as orientations defined therebetween. Because the return to the neutral orientation 366 does not rely on pushing of the distal end steering section 37 with the fiber optic 132, concerns regarding buckling of the fiber optic 132 are diminished, and the cross-section 133 of the fiber optic 132 may be reduced. Reduction of the cross-section 133 frees up space in the cross-section 167 of the distal head portion 34, thus providing more room for other components (e.g., larger irrigation channels 122) or for reduction in the overall area of the cross-section 167.

In the embodiment of FIGS. 2 through 7, the illumination fiber optic 132 is depicted and described as driving the bilateral deflection of the steering section 37. Also, the FIGS. 2 through 7 embodiment is depicted and described as preserving the integrity of the working channel 102 (e.g., providing aspiration), while the working channel 124 (e.g., providing irrigation) is breached by the plurality of gaps 312 and is in fluid communication with the sleeve 326. These functions may be performed with other components of the endoscopic system 30. For example, it is contemplated that the laser fiber optic lumen 266 and laser fiber optic 112 be disposed within the working channel 102 proximate the outer surface of the base 96 (i.e., with increased distance from the central axis 110). In such an arrangement, the laser fiber optic 112 and the single illumination fiber optic 132 could operate as push-pull linkages to steer the catheter 32. Also, configurations are contemplated where the plurality of segments 304 are configured to preserve the integrity of the irrigation channel 122 and the working channel 102 is breached and contained by the sleeve 326. Such modifications are within the grasp of the skilled artisan based on the principles presented in this disclosure.

Each of the additional figures and methods disclosed herein can be used separately, or in conjunction with other features and methods, to provide improved devices and methods for making and using the same. Therefore, combinations of features and methods disclosed herein may not be necessary to practice the disclosure in its broadest sense and are instead disclosed merely to particularly describe representative and preferred embodiments.

Various modifications to the embodiments may be apparent to one of skill in the art upon reading this disclosure. For example, persons of ordinary skill in the relevant arts will recognize that the various features described for the different embodiments can be suitably combined, un-combined, and re-combined with other features, alone, or in different combinations. Likewise, the various features described above should all be regarded as example embodiments, rather than limitations to the scope or spirit of the disclosure.

Persons of ordinary skill in the relevant arts will recognize that various embodiments can comprise fewer features than illustrated in any individual embodiment described above. The embodiments described herein are not meant to be an exhaustive presentation of the ways in which the various features may be combined. Accordingly, the embodiments are not mutually exclusive combinations of features; rather, the claims can comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Unless indicated otherwise, references to “embodiment(s)”, “disclosure”, “present disclosure”. “embodiment(s) of the disclosure”, “disclosed embodiment(s)”, and the like contained herein refer to the specification (text, including the claims, and figures) of this patent application that are not admitted prior art.

For purposes of interpreting the claims, it is expressly intended that the provisions of 35 U.S.C. 112(f) are not to be invoked unless the specific terms “means for” or “step for” are recited in the respective claim.

1. An endoscopic device, comprising:
a steering section including a plurality of segments arranged sequentially along a central axis, said plurality of segments being separated at a first lateral side of said steering section to define a plurality of gaps therebetween; and
a fiber optic that extends to a distal end portion of said steering section, wherein placing said fiber optic in tension causes said steering section to deflect in a first lateral direction.

2. The endoscopic device of claim 1, wherein said fiber optic is anchored proximate said distal end portion of said steering section.

3. The endoscopic device of claim 1, wherein said plurality of segments are joined at a second lateral side of said steering section.

4. The endoscopic device of claim 1, wherein said fiber optic is an illumination fiber optic.

5. The endoscopic device of claim 1, comprising a distal head portion attached to said distal end portion.

6. The endoscopic device of claim 5, wherein said distal head portion includes a base and a transparent cap.

7. The endoscopic device of claim 6, wherein said fiber optic is anchored to said base of said distal head portion.

8. The endoscopic device of claim 1, wherein said steering section defines a guide passage proximate said first lateral side, said fiber optic being disposed in said guide passage.

9. The endoscopic device of claim 8, wherein each of said plurality of segments defines a guide passage segment to define said guide passage, said guide passage segments being concentric about a guide axis, said fiber optic passing through said guide passage segments along said guide axis.

10. The endoscopic device of claim 9, wherein said fiber optic is a single fiber optic that passes through the guide passage segments.

11. The endoscopic device of claim 10, wherein said single fiber optic defines an oblong cross-section.

12. The endoscopic device of claim 1, wherein said steering portion defines a first working channel and a second working channel, said first working channel being breached to define said plurality of gaps.

13. The endoscopic device of claim 12, wherein said second working channel is adjacent said second lateral side.

14. The endoscopic device of claim 12, wherein said second working channel being continuous through said steering section.

15. The endoscopic device of claim 1, wherein said plurality of segments are surrounded by a flexible sleeve.

16. The endoscopic device of claim 15, wherein said sleeve is anchored to said base of said head portion.

17. The endoscopic device of claim 15, wherein said sleeve is anchored to a proximal portion of said steering section.

18. The endoscopic device of claim 1, wherein placing said fiber optic in compression causes said steering section to deflect in a second lateral direction.

19. The endoscopic device of claim 18, wherein said first lateral direction is opposite said second lateral direction.

20. The endoscopic device of claim 19, wherein:
said first lateral side is in said first lateral direction f om said central axis; and
said second lateral side is in said second lateral direction from said central axis.
